(19) United States
(12) Patent Application Publication
(10) Pub. No.: US 2024/0342148 A1
(43) Pub. Date: Oct. 17, 2024

(54) METHODS OF TREATING FACIOSCAPULOHUMERAL MUSCULAR DYSTROPHY WITH LOSMAPIMOD

(71) Applicant: Fulcrum Therapeutics, Inc., Cambridge, MA (US)

(72) Inventors: Michelle Mellion, Cambridge, MA (US); John Jiang, Cambridge, MA (US); Anthony Accorsi, Cambridge, MA (US); Alejandro Rojas, Cambridge, MA (US)

(21) Appl. No.: 18/580,024

(22) PCT Filed: Jul. 27, 2022
(86) PCT No.: PCT/US2022/038589

§ 371 Date: Jan. 17, 2024

Related U.S. Application Data
(60) Provisional application No. 63/344,844, filed on May 23, 2022.
(60) Provisional application No. 63/328,975, filed on Apr. 8, 2022.
(60) Provisional application No. 63/320,510, filed on Mar. 16, 2022.
(60) Provisional application No. 63/203,628, filed on Jul. 27, 2021.

(51) Int. Cl. A61K 31/4418; A61B 5/00; A61B 5/055; A61P 21/00

(52) U.S. Cl. CPC A61K 31/4418; A61B 5/055; A61B 5/4519; A61B 5/4848; A61P 21/00

(57) ABSTRACT

Provided herein, in part, are methods of treating facioscapulohumeral muscular dystrophy in a patient in need thereof, comprising administering losmapimod to the patient.

### CROSS-REFERENCE

This application claims priority to U.S. Provisional Patent Application No. 63/203,628 filed Jul. 27, 2021, U.S. Provisional Patent Application No. 63/320,510 filed Mar. 16, 2022, U.S. Provisional Patent Application No. 63/328,975 filed Apr. 8, 2022, and U.S. Provisional Patent Application No. 63/344,844 filed May 23, 2022, the contents of each of which are incorporated herein by reference.

### BACKGROUND

The muscular dystrophies (MD) are a group of more than 30 different genetic diseases characterized by progressive weakness and degeneration of the skeletal muscles that control movement. Some forms of MD occur in infancy or childhood, while others may not appear until middle age or older. The various MD diseases differ in terms of the distribution and extent of muscle weakness (some forms of MD also affect cardiac muscle), age of onset, rate of progression, and pattern of inheritance.

Facioscapulohumeral muscular dystrophy (FSHD) is the third most common form of muscular dystrophy. FSHD is caused by genetic mutations resulting in the epigenetic derepression of the DUX4 gene, which makes this disease unique among muscular dystrophies.

There is currently no approved treatment that can halt or reverse the effects of FSHD, although nonsteroidal anti-inflammatory drug are often prescribed to improve comfort and mobility. There is a need for new methods to treat FSHD.

### SUMMARY

The present disclosure provides, in part, methods of treating Facioscapulohumeral muscular dystrophy (FSHD) in a patient in need thereof.

In one embodiment, provided herein is a method of treating facioscapulohumeral muscular dystrophy (FSHD) in a patient in need thereof, comprising: administering 15 mg, twice daily, of losmapimod to the patient for at least 40 weeks, wherein after the at least 40 weeks the patient has improved muscle health as measured by whole-body musculoskeletal magnetic resonance imaging (WB-MSK-MRI).

In another embodiment, provided herein is a method of reducing fat accumulation in a muscle at a high risk of progression of facioscapulohumeral muscular dystrophy (FSHD), in a patient in need thereof, comprising: administering 15 mg, twice daily, of losmapimod for at least 40 weeks.

### BRIEF DESCRIPTION OF THE DRAWINGS

FIG. 1 depicts a schematic of the clinical study of Example 1.

FIG. 2 depicts the types of muscles included in regional correlation Composite Studies with respect to clinical outcome assessments.

FIGS. 3A and 3B depict exemplary pharmacokinetics and target engagement results of the study of Example 1. FIG. 3A depicts losmapimod concentrations in plasma. Red dots represent mean concentration in ng/ml and error bars Standard Error (SE). FIG. 3B depicts target engagement in blood determined by ratio of phosphorylated HSP27 and total HSP27 relative to placebo levels at each timepoint. Orange squares represent mean and error bars (SE).

FIG. 4 depicts Selected Secondary and Exploratory Efficacy End Points of the study of Example 1.

FIGS. 5A and 5B depict exemplary DUX4-driven gene expression distribution and heterogeneity results obtained from the study of Example 1, as shown by scatter plots showing each measure of DUX4-driven gene expression in muscle needle biopsies. FIG. 5A depicts combined baseline and postbaseline values (week 16 and week 36).

FIG. 5B depicts populations separated by visit, Week 16 or Week 36. Week 16, n=21 for placebo, n=24 for losmapimod. Week 36, n=17 for placebo, n=15 for losmapimod. Lines represent means per group.

FIG. 6 depicts selected secondary and exploratory efficacy end points of the study of Example 1.

FIG. 7 depicts exemplary Patient Global Impression of Change Breakdowns from the study of Example 1. Breakdown of responses are in percentage by timepoint in PGIC rating. Number of participants in each timepoint is indicated at the top.

FIG. 8 depicts exemplary reachable workspace results from the study of Example 1.

FIG. 9 depicts exemplary annualized rates of change in reachable workspace. The annualized rate of change was calculated using a linear mixed-effects model to estimate percent change per year (y-axis). As indicated in the legend, the orange line represents losmapimod and the blue line represents placebo. Light lines represent standard errors for losmapimod and placebo.

FIG. 10 depicts exemplary dynamometry results of the study of Example 1.

FIG. 11 depicts other exemplary endpoints of the study of Example 1, including FSHD-TUG, MFM, and FSHD-HI.

FIG. 12A depicts a distribution of the echogenicity z-scores (<2, 2-4, 4-6, and >6) of muscles in FSHD patients receiving losmapimod at baseline versus at Week 60. FIG. 12B depicts the change from baseline to Week 60 in echogenicity of multiple muscles and muscle groups in FSHD patients receiving 15 mg twice daily losmapimod.

FIG. 13A depicts the correlation of echogenicity z-score of the upper extremities vs Reachable Workspace in FSHD patients at Baseline. FIG. 13B depicts the correlation of echogenicity z-score of the upper extremities vs Reachable Workspace in FSHD patients receiving 15 mg twice daily losmapimod at Week 60.

FIG. 14A depicts the correlation of echointensity of the Tibialis Anterior to Handheld Dynamometry for Ankle Dorsiflexion data in FSHD patients at Baseline. FIG. 14B depicts the correlation of echointensity of the Tibialis Anterior to Handheld Dynamometry for Ankle Dorsiflexion data in FSHD patients receiving 15 mg twice daily losmapimod at Week 60.

FIG. 15A depicts the correlation of echointensity of the lower extemities vs Timed Up-And-Go (TUG) in FSHD patients at Baseline. FIG. 15B depicts the correlation of echointensity of the lower extemities vs TUG in FSHD patients receiving 15 mg twice daily losmapimod at Week 60.

FIG. 16 depicts a schematic of the wearable sensor study of Example 4 in FSHD patients receiving 15 mg twice daily losmapimod.

FIG. 17 depicts feasibility and compliance results for FSHD patients in the wearable sensor study of Example 4.

FIG. 18 depicts analysis, processing and reliability results for FSHD patients in the wearable sensor study of Example 4.

FIG. 19 depicts correlation data between clinical and wearable sensor variables for FSHD patients in the wearable sensor study of Example 4.

FIG. 20 depicts results of disability location results between in-clinic and wearable variables in the wearable sensor study of Example 4.

FIG. 21 depicts physical function as measured by gait velocity over a 1 year losmapimod treatment period in FSHD patients, as categorized by the groups of FIG. 20, in the wearable sensor study of Example 4.

FIG. 22 depicts updated annualized rates of change in total RSA with weight in FSHD patients receiving 15 mg twice daily losmapimod in the study of Example 1.

FIG. 23 depicts annualized rates of change in RSA by domains, including with use of weights and without weights and total RSA in patients receiving 15 mg twice daily losmapimod in the study of Example 1.

FIG. 24 depicts annualized rates of change in MFI, annualized rates of change in MFF, and annualized rates of change in LMV in patients receiving 15 mg twice daily losmapimod in the study of Example 1.

FIG. 25 depicts annualized rates of change in TUG Average Completion Time in patients receiving 15 mg twice daily losmapimod in the study of Example 1.

FIG. 26 depicts change in maximal voluntary isometric contraction testing (MVICT) for handgrip and shoulders in patients receiving 15 mg twice daily losmapimod in the study of Example 1.

FIG. 27 depicts results from an Exploratory Annualized Reachable Workspace (RWS) Analysis in the Open Label Study (OLS) of Example 1.

### DETAILED DESCRIPTION

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure.

### Definitions

“Individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all enantiomeric, diastereomeric, and geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure.

“Therapeutically effective amount” includes the amount of the subject compound that will elicit the biological or medical response of a tissue, system, animal or human that is being sought by the researcher, veterinarian, medical doctor or other clinician. A compound described herein, e.g., a p38α/β MAPK inhibitor described herein, is administered in therapeutically effective amounts to treat a condition, e.g., a condition described herein. Alternatively, a therapeutically effective amount of a compound is the quantity required to achieve a desired therapeutic and/or prophylactic effect, such as an amount which results in the prevention of or a decrease in the symptoms associated with the condition.

As used herein, “Wk” refers to week.

A compound described herein, e.g., a p38α/β MAPK inhibitor described herein, can be formulated as a pharmaceutical composition using a pharmaceutically acceptable carrier and administered by a variety of routes. In some embodiments, such compositions are for oral (PO) administration. In some embodiments, such compositions are for parenteral (by injection) administration. In some embodiments, such compositions are for transdermal (TD) administration. In some embodiments, such compositions are for intravenous (IV) administration. In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19th ed., Mack Publishing Co., 1995).

### Methods of Use and Treatment

The present disclosure also provides, in another embodiment, methods of treating of facioscapulohumeral muscular dystrophy (FSHD) in a patient in need thereof, comprising administering to the patient a therapeutic agent described herein, e.g., a p38a/β MAPK inhibitor. In certain embodiments, the p38a/B MAPK inhibitor is a compound of Formula (I):

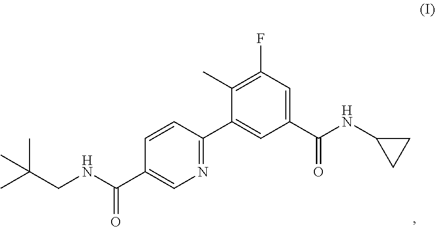 [SMILES: Cc1c(F)cc(C(=O)NC2CC2)cc1-c1ccc(C(=O)NCC(C)(C)C)cn1]

or a pharmaceutically acceptable salt thereof.

In another embodiment, provided herein is a method of treating facioscapulohumeral muscular dystrophy (FSHD) in a patient in need thereof, comprising: administering 15 mg, twice daily, of losmapimod to the patient for at least 40 weeks, wherein after the at least 40 weeks the patient has improved muscle health as measured by whole-body musculoskeletal magnetic resonance imaging (WB-MSK-MRI).

In another embodiment, provided herein is a method of reducing fat accumulation in a muscle at a high risk of progression of facioscapulohumeral muscular dystrophy (FSHD), in a patient in need thereof, comprising: administering 15 mg, twice daily, of losmapimod for at least 40 weeks.

In some embodiments, after the at least 40 weeks the muscle has reduced fat accumulation as measured by whole-body musculoskeletal magnetic resonance imaging (WB-MSK-MRI). In some embodiments, the method comprises imaging the muscle using whole-body musculoskeletal magnetic resonance imaging (WB-MSK-MRI) to determine a correlation between a measure of muscle health and a clinical outcome assessment of the patient. In some embodiments, the measure of muscle health is selected from the group consisting of muscle fat fraction (MFF), muscle fat infiltration (MFI), and lean muscle volume (LMV). In some embodiments, the clinical outcome assessment is selected from the group consisting of a reachable workspace (RWS), a relative surface area (RSA), and an assessment made from a Timed Up and Go (TUG) test. In some embodiments, the muscle, prior to administering losmapimod to the patient, is characterized by a muscle fat infiltration (MFI) of less than about 0.10 and a muscle fat fraction (MFF) of less than about 0.50. In some embodiments, the muscle, prior to administering losmapimod to the patient, is characterized by a muscle fat infiltration (MFI) of greater than or equal to about 0.10 and a muscle fat fraction (MFF) of less than about 0.50. In some embodiments, the muscle, prior to administering losmapimod to the patient, is characterized by a muscle fat fraction (MFF) of greater than or equal to about 0.50. In some embodiments, the method comprises administering 15 mg, twice daily, of losmapimod to the patient for at least 48 weeks. In some embodiments, the FSHD is FSHD1. In some embodiments, the muscle is selected from the group consisting of a muscle of an upper extremity and a muscle of a lower extremity. In some embodiments, the muscle is selected from the group consisting of shoulder abductor and an ankle dorsiflexor. In some embodiments, the shoulder abductor is selected from the group consisting of a bilateral shoulder abductor, a dominant shoulder abductor, and a non-dominant shoulder abductor.

In other embodiments, the treatment of a patient in the methods described herein is evaluated by an assessment of the patient including, but not limited to, reachable workspace quantification of total relative surface area (Q1-Q5) (e.g., with 500 g wrist weight in the dominant arm), quality of life in the neurological disorders upper extremity scale (Neuro-QOL UE), patient global impression of change (PGIC), and muscle ultrasound. In some embodiments, the treatment of a patient in the methods described herein is evaluated by the Functional Assessment of Chronic Illness Therapy (FACIT). In some embodiments, said method comprises administering to the patient in need thereof 15 mg of losmapimod twice daily.

In some embodiments, the treatment of a patient in the methods described herein is evaluated by a patient reported outcome (PRO). In some embodiments, the PRO is a PRO of physical function. Exemplary physical functions measured through a PRO described herein include, but are not limited to, mobility, upper extremity dexterity, daily activities, facial function (e.g., smiling, verbal communication), central/axial function, pain, fatigue, upper extremity range of motion, upper extremity weakness, core weakness, lower extremity weakness, facial weakness, and foot drop.

### Compounds

In an embodiment, methods of the present disclosure comprise administration of a therapeutically effective amount of a compound of Formula (I) (also referred to herein as “losmapimod”). Losmapimod is an inhibitor of p38α/β MAPK, and has the chemical name 6-(5-cyclopropylcarbamoyl-3-fluoro-2-methyl-phenyl)-N-(2,2-dimethylpropyl-)-nicotinamide and the structure:

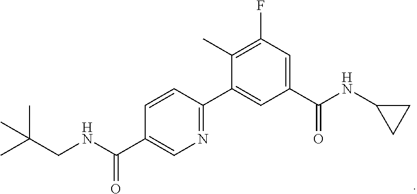 [SMILES: Cc1c(F)cc(C(=O)NC2CC2)cc1-c1ccc(C(=O)NCC(C)(C)C)cn1]

Suitable methods for preparing losmapimod are disclosed, for example, in U.S. Pat. No. 7,125,898.

### EXAMPLES

The examples described below are offered to illustrate the compounds, pharmaceutical compositions and methods provided herein and are not to be construed in any way as limiting their scope.

### Example 1. A Randomized, Double-Blind, Placebo-Controlled, 48-Week Study of the Efficacy and Safety of Losmapimod in Treating Facioscapulohumeral Muscular Dystrophy

This is a Phase 2b, randomized, double-blind, placebo-controlled, 48-week, international, parallel-group study (RCT) of the efficacy and safety of losmapimod in subjects with FSHD, with Open Label Extension (OLE), was designed and conducted to evaluate the efficacy of losmapimod in treating FSHD.

The trial is being conducted in two parts: a RCT treatment period and an OLE period (all subjects treated with losmapimod). The RCT period evaluated the efficacy and safety of losmapimod in FSHD subjects for up to 48 weeks. The OLE period is ongoing.

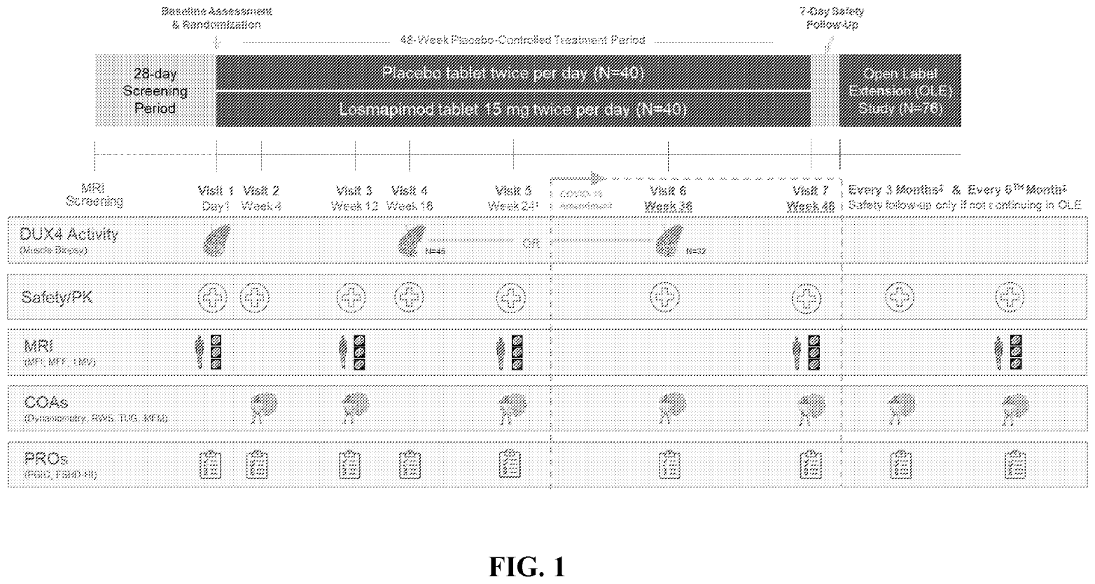

Eighty subjects were randomized 1:1 to receive 15 mg tablets of losmapimod or placebo orally BID. The RCT period was intended to last 24 weeks; due to the COVID-19 pandemic, it was amended to 48 weeks. The OLE period is ongoing until marketing approval or the Sponsor halts the study (FIG. 1). Seventy-seven subjects completed the RCT period. Sixteen subjects entered into the OLE after completing the Wk24 visit (prior to COVID19 amendment); 60 subjects entered the OLE after the Wk48 visit. One subject declined OLE after completing the RCT. General inclusion criteria were: age 18-65 years, confirmed diagnosis of FSHD1, Ricci score 2-4 at screening, and a STIR+ muscle, as determined by a central reader, safely accessible by needle biopsy with MRI.

The primary endpoint was change in DUX4 expression, as evaluated by a composite measure of selected DUX4-regulated gene transcripts in skeletal muscle. Secondary endpoints included safety and tolerability (AEs), prespecified WB-MSK-MRI composite scores of Muscle Fat Infiltration (MFI), Muscle Fat Fraction (MFF), and Lean Muscle Volume (LMV) in muscles at high risk of progression (classified as B muscles), and PK (plasma and muscle)/target engagement (blood). Exploratory endpoints were evaluation of change in two PROs (Patients' Global Impression of Change [PGIC] Questionnaire and the FSHD-Health Index [FSHD-HI]) and FSHD relevant COAs including reachable workspace (RWS), a 3D motion sensor-based outcome assessment measuring individual global upper extremity function, including shoulder and proximal arm; Timed Up and Go [TUG], which measures how long it takes a subject to rise from a seated position, walk 3 m, then return to the chair; the FSHD-TUG; muscle strength as measured by hand-held manual dynamometry (shoulder abduction, elbow flexion/extension, ankle dorsiflexion, and hand grip), and Motor Function Measure Domain 1 (MFM).

All randomized subjects were included in the efficacy analysis, in which subjects were analyzed according to randomized treatment (full analysis set). Efficacy endpoints with only 1 post-baseline follow-up, such as the primary endpoint, were analyzed using the analysis of covariance (ANCOVA) approach. Efficacy endpoints with two or more post-baseline follow-ups, such as secondary and exploratory efficacy endpoints, were analyzed using a mixed-effects model for repeated measures (MMRM). Hierarchical hypothesis testing started with the primary endpoint, then LMV, then FSHD-TUG. Other secondary and exploratory analyses were pre-specified without hierarchical prioritization.

### Methods

### Ricci Score

Ricci score is a well-established metric of patient disability. The score ranges from 0-10, where 0 indicates no muscle weakness, and 10 indicates wheelchair dependency.

### Statistical Methods

Assuming an effect size of 0.70, a sample size of 68 subjects (34 subjects per group) was considered necessary to provide 80% power with a 2-sided test at a 0.05 significance level to detect a difference between losmapimod and placebo in change from baseline in DUX4 activity in affected skeletal muscle after 16 weeks or 36 weeks (depending upon when the muscle biopsy was performed) during the placebo-controlled treatment period. Assuming that approximately 10% of subjects would be non-evaluable, approximately 76 subjects were required to be randomly assigned at a 1:1 ratio to losmapimod and placebo (38 subjects per group).

Subjects were randomly assigned at the baseline visit (Day 1) to receive losmapimod (active drug) or placebo using a 1:1 allocation ratio. Randomization was stratified to ensure that the treatment allocation was balanced across FSHD repeat number categories (i.e., 1 to 3 repeats versus 4 to 9 repeats). An Interactive Response Technology (IRT) system was used to administer study drug according to the randomization schedule.

All efficacy analyses were performed using the full analysis set (FAS), according to the randomized treatment group. The primary endpoint, change from baseline in DUX4 activity (DUX4 Score 1), was analyzed using the analysis of covariance approach, with treatment group and FSHD repeat number category (1 to 3 repeats versus 4 to 9 repeats) as fixed effects, and baseline DUX4 Score 1 as a covariate. Multiplicity adjustment was not performed, as the primary endpoint was not met.

Continuous secondary and exploratory efficacy endpoints with 2 or more postbaseline follow-ups were analyzed using a mixed-effects model for repeated measures (MMRM), with change from baseline as the dependent variable, and treatment group, visit, treatment group-by-visit interaction, and FSHD repeat number category as fixed effects, and baseline value as a covariate. An unstructured covariance matrix was used to model the correlations among repeated measurements within each subject. The Kenward-Roger approximation was used to estimate denominator degrees of freedom.

The RWS RSA score was also analyzed using a linear mixed-effects model, with treatment group as a fixed effect, and intercept, time and treatment group-by-time interaction as random effects, and with adjustment for FSHD repeat number category and region (USA, Canada, and EU). An unstructured covariance matrix was used.

The placebo-controlled treatment period was performed in a double-blind fashion. The investigator, study staff, subjects, sponsor, and monitor remained blinded to the treatment until study closure.

The testing hierarchy for primary and key secondary analyses was ordered as follows: change from baseline in DUX4 activity (DUX4 Score 1) at Week 16 or 36 combined, change from baseline in longitudinal whole-body LMV composite score at Week 48, and change from baseline in average completion time of FSHD TUG at Week 48; each being tested at 5% significance level. Other secondary and exploratory endpoints were analyzed as prespecified (unless otherwise noted) without hierarchical prioritization.

All safety analyses were performed using the safety analysis set based on as-treated treatment group. Safety analyses included summaries of TEAEs, clinical laboratory, vital signs and ECGs.

### Muscle Needle Biopsies

Muscle needle biopsies were performed using the Bergstrom needle or fine needle at D1 and Wk16, or Wk36 if the muscle biopsy could not be performed at Wk16. The choice of the muscle(s) to be biopsied was determined by the investigator, informed by MRI taken during screening. Only those STIR+ muscles with 10%≤MFF≤40% were eligible for biopsy. Previous studies demonstrated an increased probability of detecting DUX4 activity in STIR+ muscles. The bilateral vastus lateralis, vastus medialis, lateral gastrocnemius, medial gastrocnemius, and tibialis anterior were evaluated for eligibility by MRI using a central reader. The Wk16 or Wk36 muscle needle biopsies were performed in the same approximate location as the pretreatment (D1) muscle biopsy. At baseline, MRI fiducials were placed in order to identify the location for the needle biopsy. Coordinates in reference to the closest fiducial defined the location for the muscle biopsy. A grid was used to record the placement of the fiducials, so their location (and hence the area to biopsy) could be identified at Wk16 or Wk36 timepoint. Approximately 15-40 mg of tissue was obtained at each biopsy and frozen in liquid nitrogen within 60 seconds after excision and stored at −80° C. (SciSafe Inc. Billerica, MA, USA) until analysis.

### DUX4-Regulated Transcript Analysis by RT-qPCR in Muscle Biopsies

The muscle tissue collected at each muscle needle biopsy (D1 and Wk16 or Wk36) was analyzed for DUX4 activity using a molecular panel of DUX4-regulated gene transcripts (CCNA1, KHDC1L, MBD312, PRAMEF6, SLC34A2 and ZSCAN4, with TBP, HMBS, CDKN1B as reference genes). Raw Cycle Thresholds (Cts) from quantitative polymerase chain reaction (qPCR) were determined for DUX4 regulated genes using a validated assay (Fluidigm). The raw Cts per gene per sample (typically replicated) were normalized to reference gene values by a blinded scientist. Reference genes were confirmed to have coefficient of variation <30% for all pairs of genes. The sample-analyte average Ct value (arithmetic mean by analyte and by sample) represents the relative abundance level for that analyte in the assayed sample. The arithmetic mean of the sample-analyte average for each reference gene value (geometric average of the signal) is the sample reference value used by all the target analytes for that sample. The Inverted Delta Ct value for each sample/analyte is the difference between 30 and the difference of the target analyte Ct and the reference Ct value. The first difference is used as 30 is usually the max cycle and allows for inversion of the Delta Ct value. The mean of the Inverted Delta Ct values across all 6 transcripts, by subject, by timepoint, is the DUX4-driven gene expression (primary endpoint).

### WB-MSK MRI

The images were acquired in five different sections. The rotator cuff was imaged using a neck coil, torso and legs using anterior surface coils and integrated table coils, and the upper extremities using a surface and table coils, after the patient was repositioned such that the extremity was in the center of the MRI scanner.

T1-weighted Dixon-images of 18 muscles bilaterally, 36 total, were acquired and water and fat images were reconstructed using the scanner built-in phase-sensitive reconstruction (Siemens: Dixon-Vibe; Philips: mDixon). Three different measures were calculated for each muscle. The muscle fat fraction (MFF) is the total fat fraction inside the muscle fascia. The muscle fat infiltration (MFI) represents the diffuse fat infiltration in the muscle tissue and is defined as the fat fraction of the muscle voxels that contain <50% fat. Therefore, MFI measures the MFF for muscle tissue that is not end-stage. The lean muscle volume (LMV) represents the total amount of functional muscle tissue and is measured by removing all fat within the muscle volume. Each muscle was categorized based on these measures and supported by prior literature as normal appearing (Category A, MFI<0.10; MFF<0.50), intermediate (Category B, MFI≥0.10; MFF<0.50), and late-stage (Category C, MFF≥0.50).

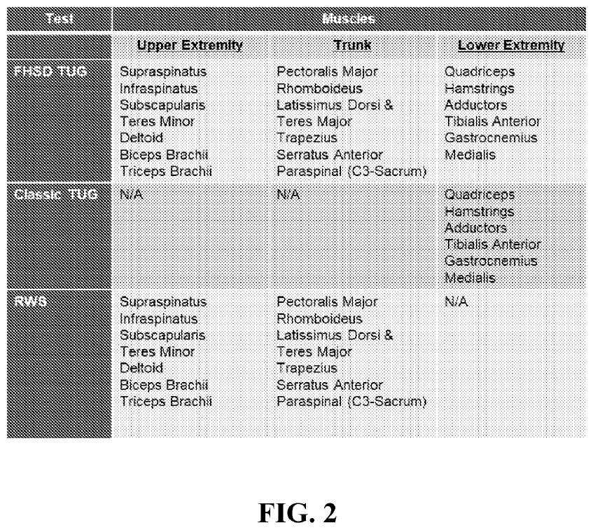

Two composite scores were used to assess the treatment efficacy and correlation with relevant clinical outcome assessments. The treatment efficacy composite score includes only those muscles identified as normal appearing or at high risk for progression (Category A or B muscles) at baseline that were measurable at both observation timepoints, having no signal quality issues. The regional composite scores for correlations are composed of all muscles that were involved in the specific functional assessment of interest regardless of their categorization (FIG. 2). The scores were derived by scientists at the MRI Service Provider (AMRA Medical Inc.) in a blinded fashion, with no access to treatment assignment information. Measurements for muscles with major quality issues, (i.e., muscles with missing values), were imputed. Late-stage muscles were not included in the analysis as these contain high fat fraction which makes them challenging to quantify accurately, and likely are already not functional.

A mixed-effects model for repeated measures (MMRM) was used to analyze the change from baseline in each composite MRI score (MFFₜₒₜₐₗ, LMVₜₒₜₐₗ, and MFIₜₒₜₐₗ), with repeat number category, treatment group, visit, and treatment-by-visit interactions as fixed effects and baseline value of the parameter as a covariate. Within-group LS mean changes from baseline, the associated SEs and 2-sided 95% CIs, treatment differences in LS mean changes from baseline at Weeks 12 and 48 and the associated 2-sided 95% CIs and 2-sided p-values were derived from the MMRM.

### Pharmacokinetics and Target Engagement

Blood samples for PK assessment (plasma losmapimod concentrations) were collected at D1, Wk4, Wk16, and Wk36 at the following time points: immediately pre-dose and 4 hours (±30 minutes) after administration of the study dose (approximate Cₘₐₓ). PK samples were also taken, when feasible, after dosing during the Wk12, Wk24, and Wk48 visits, preferably ≥1 hour after dosing.

Plasma and muscle losmapimod concentrations were measured by 2 validated bioanalytical high-performance liquid chromatography methods by PPD, USA (PPD 2019).

Blood samples for target engagement were collected at D1 and Wk16 or Wk36, at the same pre- and post-dose time points as PK samples.

Blood samples for measurement of total heat shock protein 27 (HSP27ₜₒₜₐₗ) and phosphorylated heat shock protein 27 (pHSP27) were collected into EDTA-containing tubes. Two mL of whole blood was stimulated ex vivo with sorbitol for 30 minutes at room temperature to induce phosphorylation of HSP27 as a measure of p38a/b MAPK activity. Sorbitol stimulation activates the p38a/b MAPK pathway in blood and allows for more robust detection of inhibition of the p38a/b MAPK pathway by losmapimod. Subsequently, samples were lysed on ice and lysates frozen at −80° C. pHSP27 and HSP27ₜₒₜₐₗ were measured by a validated enzyme-linked immunosorbent assay method (Cambridge Biomedical Inc., Boston, MA, USA and Immunologix, Tampa, FL, USA). Inter- and intra-assay precision for both pHSP27 and HSP27ₜₒₜₐₗ met the preapproved criteria of <25% CV having been run in 5 replicates with the LLOQ established for pHSP27 at 20.7 ng/ml and ULOQ at 470.1 ng/ml and the LLOQ established for HSP27ₜₒₜₐₗ at 105.2 ng/ml and ULOQ at 2168.1 ng/mL.

### Clinical Outcome Assessments (COA)

All clinical outcome assessments were administered by highly trained physical therapists.

### Reachable Work Space

The RWS was performed at all visits, except for Week 16. It uses a single, 3D sensor-based system (Microsoft Kinect) that can unobtrusively detect subjects' RWS and reflects individual global upper extremity (UE) function, including shoulder and proximal arm. RWS has high reliability, repeatability, face validity, feasibility, sensitivity to change, and promise as a clinical outcome assessment (COA) for FSHD and other neuromuscular disorders.

During the evaluation, patients were seated in front of the Microsoft Kinect sensor and underwent a standardized UE movement protocol while looking at a TV monitor. The evaluation was performed with and without 500 g weights two times per visit. All sites were provided and trained with the same standardized software and hardware by the sponsor. A central reader was responsible for training, quality control, data analysis, and standardization of the RWS across all sites in the study.

### Timed Up and Go

TUG assessments were performed at all visits, except for Wk16. The TUG test is a simple test that is used to assess a subject's mobility and requires both static and dynamic balance. TUG is a validated instrument that measures the time that a person takes to rise from a chair, walk 3 meters, turn around, walk back to the chair, and sit down. Subjects used their regular footwear, and their customary walking aid (no walkers allowed). Patients were also timed in a modified test (FSHD TUG), where they started from a supine position, then sat up, completed the TUG, and lay down again.

### Dynamometry

Quantitative isometric dynamometry strength assessments were performed at all visits, except for Week 16. The MicroFET2 hand-held dynamometer was used to measure strength in the bilateral shoulders, elbow flexors and extensors, and ankle dorsiflexors. The Jamar Plus Digital Hand Dynamometer was used to measure bilateral grip strengths. Standard physical therapy techniques were used.

### Motor Function Measure Domain 1

MFM domain 1 assessments were performed at all visits, except for those on Wk4 and Wk16. The MFM scale assesses the severity of motor deficit. Domain 1 of the MFM provides an assessment of functional impairment for standing and transfers.

FSHD-HI

The FSHD health index questionnaire was administered at all visits except for Wk16. This is a FSHD-specific patient-reported measure of disease burden on activities of daily living, quality of life, and symptom prevalence and severity. It consists of a questionnaire with 116 items developed from qualitative interviews of patients followed by a national cross-sectional validation study. The measure consists of 14 subscales that measure a patient's perception of their ambulation and mobility, hand function, shoulder and arm function, emotional health, back/chest/abdomen strength, fatigue, pain, eating function, ability to do activities, communication ability, satisfaction in social situations, performance in social situations, body image and cognition. Scoring was performed centrally by the developer.

### PGIC

The Patients' Global Impression of Change (PGIC) Questionnaire was administered at every visit after baseline. Subjects were asked to rate their overall status since the start of the study on a scale from 1 (very much improved) to 7 (very much worse). The PGIC scale has been validated in several other indications and is recommended by the FDA for use as a meaningful measure of within-patient change.

### Results

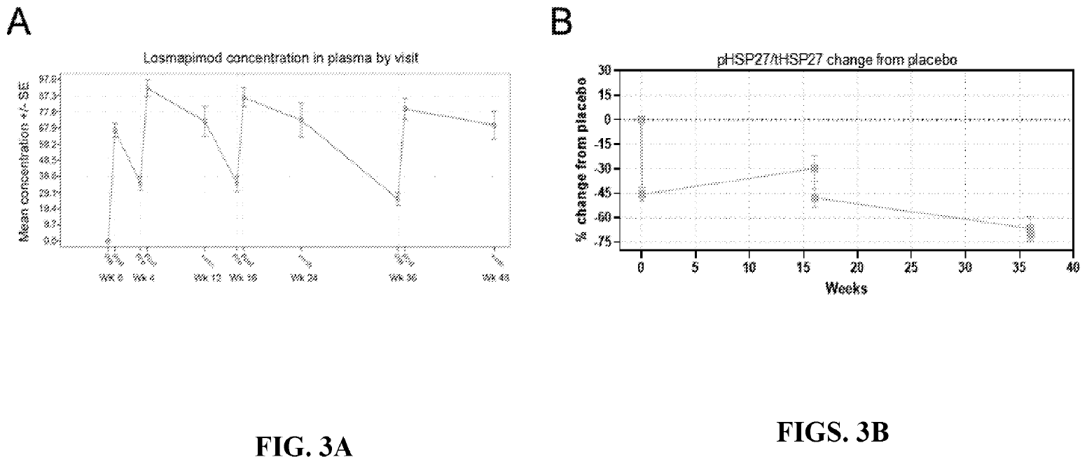

Plasma concentrations of losmapimod were consistent with previous studies at 25.2-34.4 ng/ml pre-dose, and 66.4-91.8 ng/ml 4 h post-dose (approximate Cₘₐₓ). Concentrations in muscles were also within the expected range for clinical efficacy, 61.0±7.7 ng/g at Wk16, and 91.1±10.7 ng/g at Wk36. Levels of phosphorylated heat shock protein 27 (pHSP27)/total heat shock protein 27 (HSP27ₜₒₜₐₗ), showed a reduction of 48.0-70.2% compared to placebo at Cₘₐₓ, confirming target engagement (FIGS. 3A and 3B).

Additionally, losmapimod PK profile demonstrates good adherence to study regimen. Pill counts suggest treatment compliance was >80%.

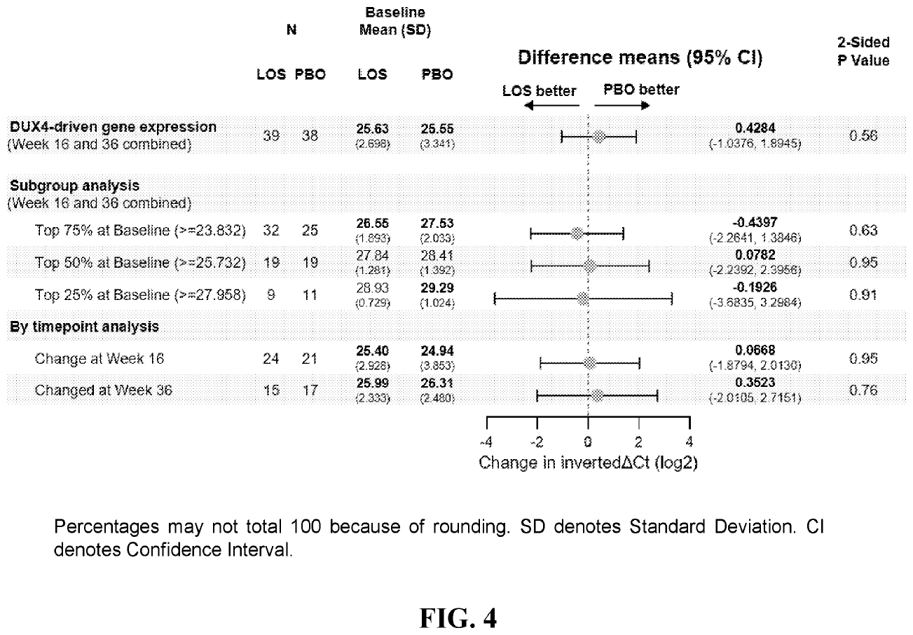

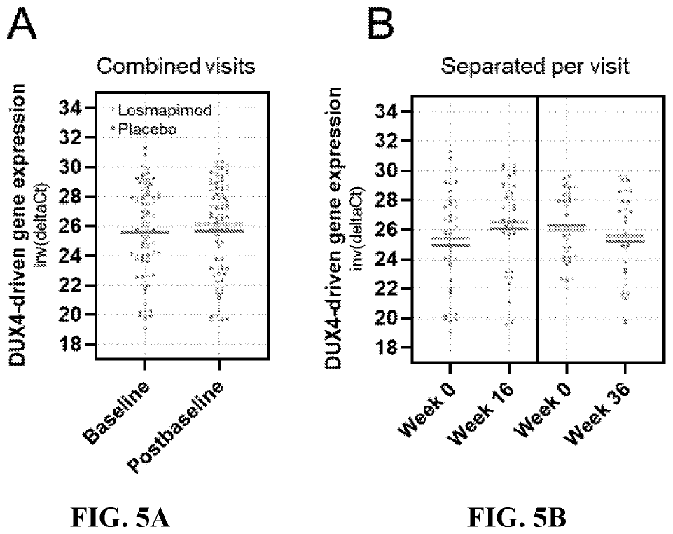

No change in DUX4 activity was noted in either placebo or losmapimod group (baseline compared to combined data of all samples from Wk16 or Wk36), and there was no difference between the groups (losmapimod 0.83, placebo 0.40, difference 0.43, 95% CI −1.04, 1.89; p=0.56; log 2 scale). Prespecified subgroup analyses by DUX4-driven gene expression did not show differences between losmapimod and placebo (FIG. 4). Significant variability in DUX4 activity was observed (pre- and post-dose) in both groups (FIGS. 5A and 5B).

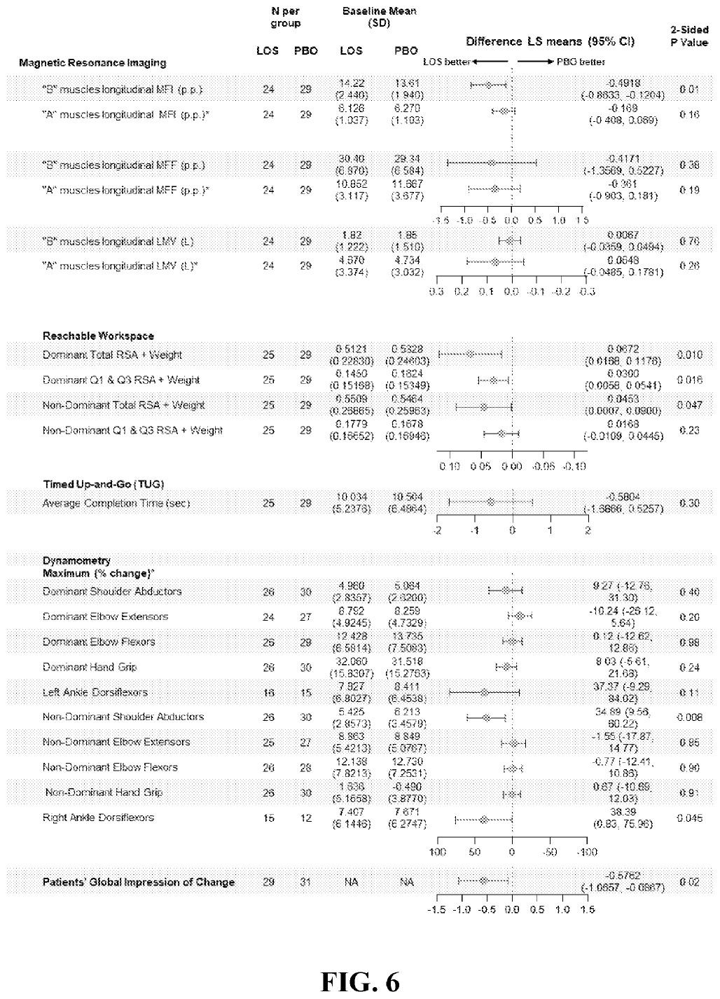

The prespecified efficacy composite measure on MFI showed significantly less fat infiltration (p=0.01) in the losmapimod group as compared to placebo in muscles at high risk of progression (classified as B muscles). There was a nonsignificant reduction of fat replacement on MFF, and no difference in LMV. A post-hoc analysis of normal appearing muscles at baseline (classified as A muscles) showed little or no accumulation of fat on MFI or MFF in the losmapimod group compared to placebo (FIG. 6).

Regional MRI composites of muscles involved in the specific functional assessment of interest demonstrated moderate and strong cross-sectional correlations for MFI, MFF, and LMV with TUG, FSHD-TUG, and RWS at individual time points throughout the study with one exception: dominant total reachable surface area (RSA) with weights in the placebo group. Table 1 shows a Spearman correlation analysis between metrics of regional correlation and composites and clinical outcome assessments at week 48. In Table 1, “LOS” refers to losmapimod and “PBO” is placebo.

TABLE 1
Spearman Correlations Between MRI metrices
and clinical outcome assessments (CoAs).

| | MFI | | MFF | | LMV | |
|---|---|---|---|---|---|---|
| | LOS | PBO | LOS | PBO | LOS | PBO |
| Dominant | −0.63 | −0.11 | −0.66 | −0.36 | 0.74 | 0.67 |
| Total RSA | | | | | | |
| with weight | | | | | | |
| p-value | (0.0018) | (0.5659) | (0.0008) | (0.0603) | (0.0001) | (0.0001) |
| N | 22 | 28 | 22 | 28 | 22 | 28 |
| Non-Dominant | −0.70 | −0.33 | −0.77 | −0.55 | 0.62 | 0.61 |
| Total RSA | | | | | | |
| with weight | | | | | | |
| p-value | (0.0003) | (0.0851) | (<0.0001) | (0.0023) | (0.0023) | (0.0006) |
| N | 22 | 28 | 22 | 28 | 22 | 28 |
| TUG | 0.38 | 0.69 | 0.48 | 0.68 | −0.42 | −0.60 |
| p-value | (0.0800) | (0.0001) | (0.0229) | (0.0001) | (0.0492) | (0.0007) |
| N | 22 | 28 | 22 | 28 | 22 | 28 |
| FSHD TUG | 0.60 | 0.51 | 0.70 | 0.70 | −0.51 | −0.62 |
| p-value | (0.0054) | (0.0049) | (0.0007) | (<0.0001) | (0.0218) | (0.0004) |
| N | 20 | 29 | 20 | 29 | 20 | 29 |

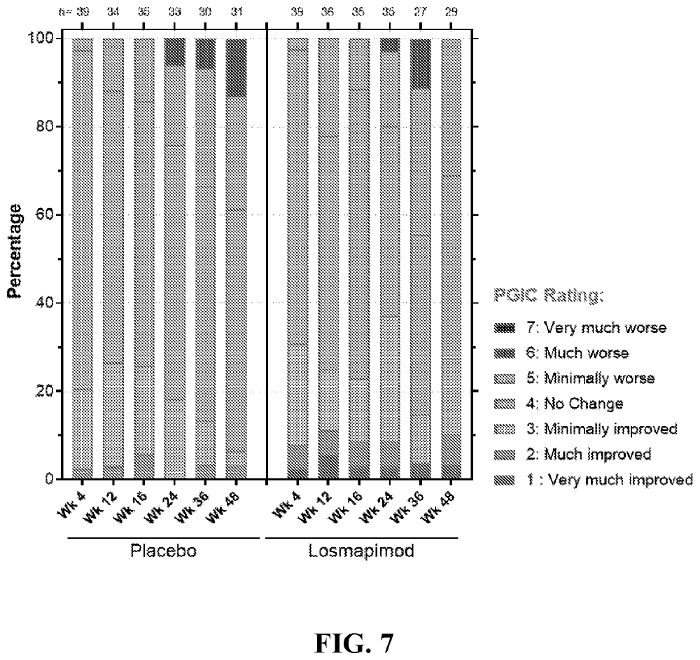

At Wk48, the losmapimod group reported significant improvement (difference 0.58 on 1-7 Likert scale; p=0.02) compared to placebo group (FIG. 6). 27.5% of losmapimod and 6.4% of placebo patients reported improvement (1-3 in Likert scale). No losmapimod treated patients reported feeling “very much worse” (6-7 in Likert scale) while 12.9% of placebo subjects did. Subjects in the placebo arm reported increasing worsening during RCT, indicating PGIC captured progression of disease (FIG. 7).

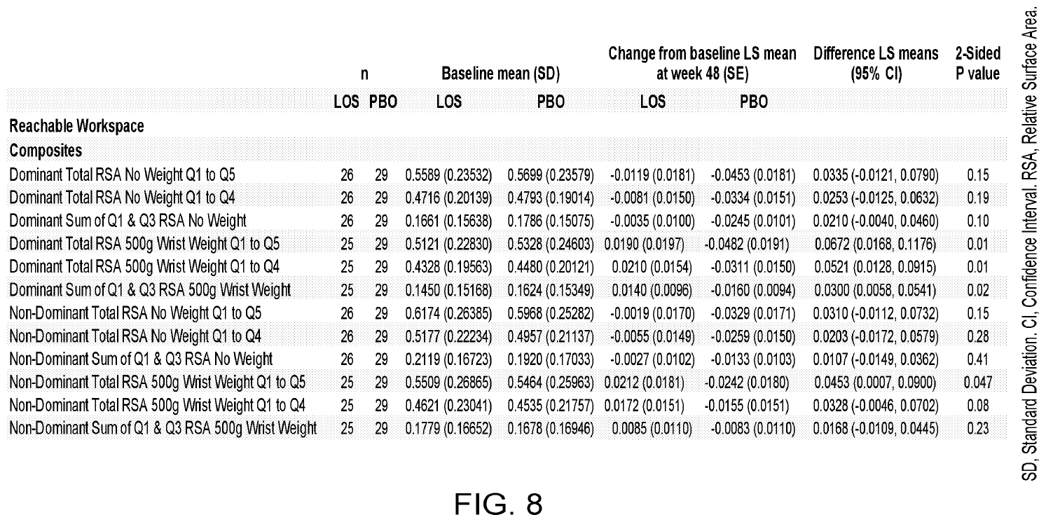

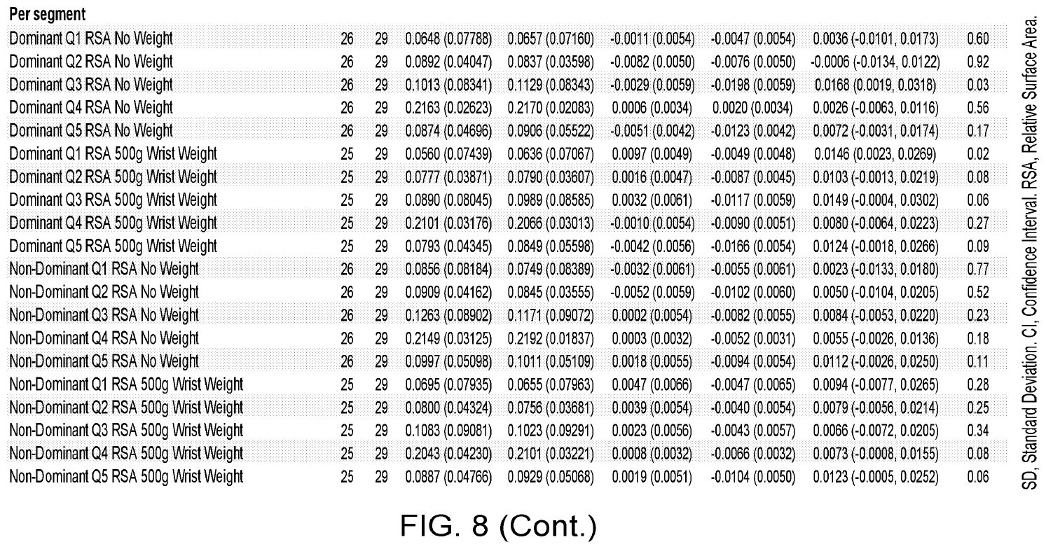

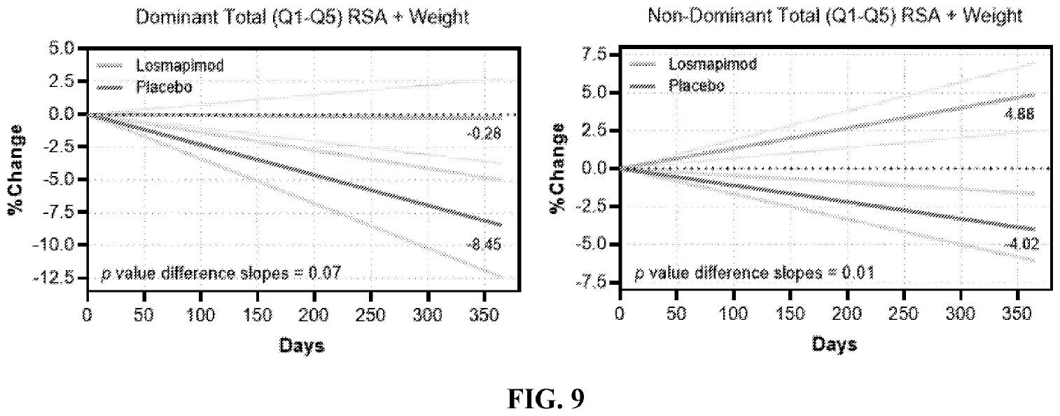

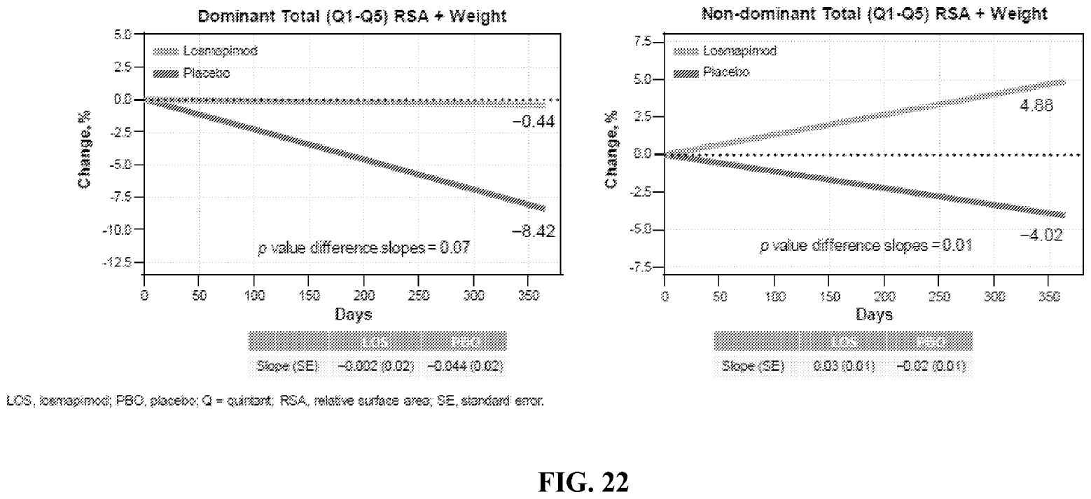

Losmapimod resulted in significant improvements in RSA, measured on a 0-1.25 scale, (dominant arm: 0.019 vs. placebo −0.048; difference 0.067, 95% CI 0.017, 0.118; p=0.01; non-dominant arm: 0.021 vs. −0.024, difference 0.045, 95% CI 0.0007, 0.09; p<0.05) (FIG. 6). Placebo subjects lost 2.6-3.6% total RSA without weights and 1.9-3.8% with weights in RWS, capturing disease progression (FIG. 8). Annualized rate of change in total RSA also showed improvements in weighted assessment (dominant, annual percent change: 0.28 vs. 8.45; p=0.07; non-dominant, 4.88 vs. −4.02; p=0.01) (FIG. 9). Updated annualized rates of change in total RSA, as shown in FIG. 22, also demonstrate that losmapimod shows slowing of disease progression or improvement in function in total RSA (Q1-Q5) with weight.

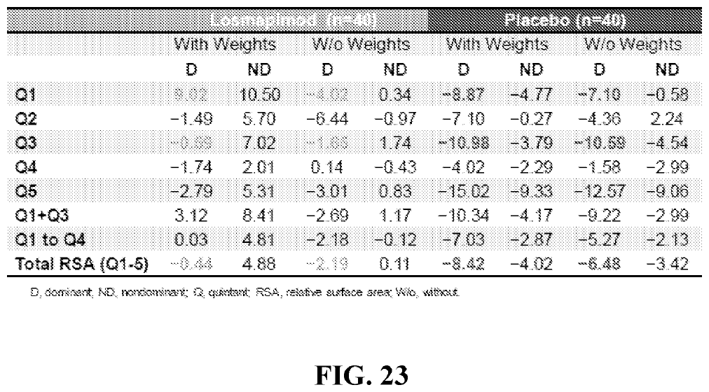

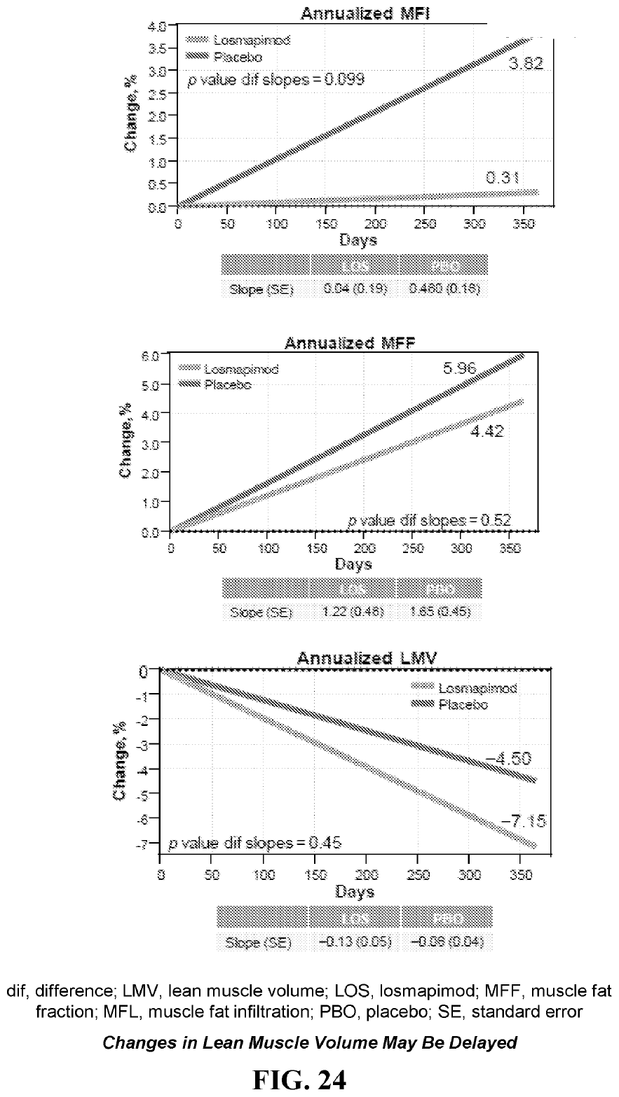

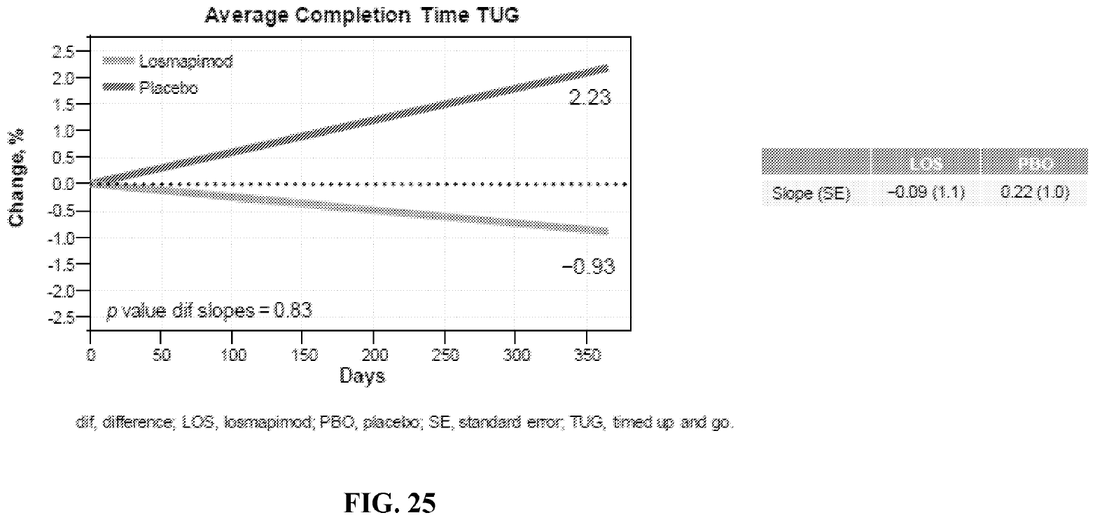

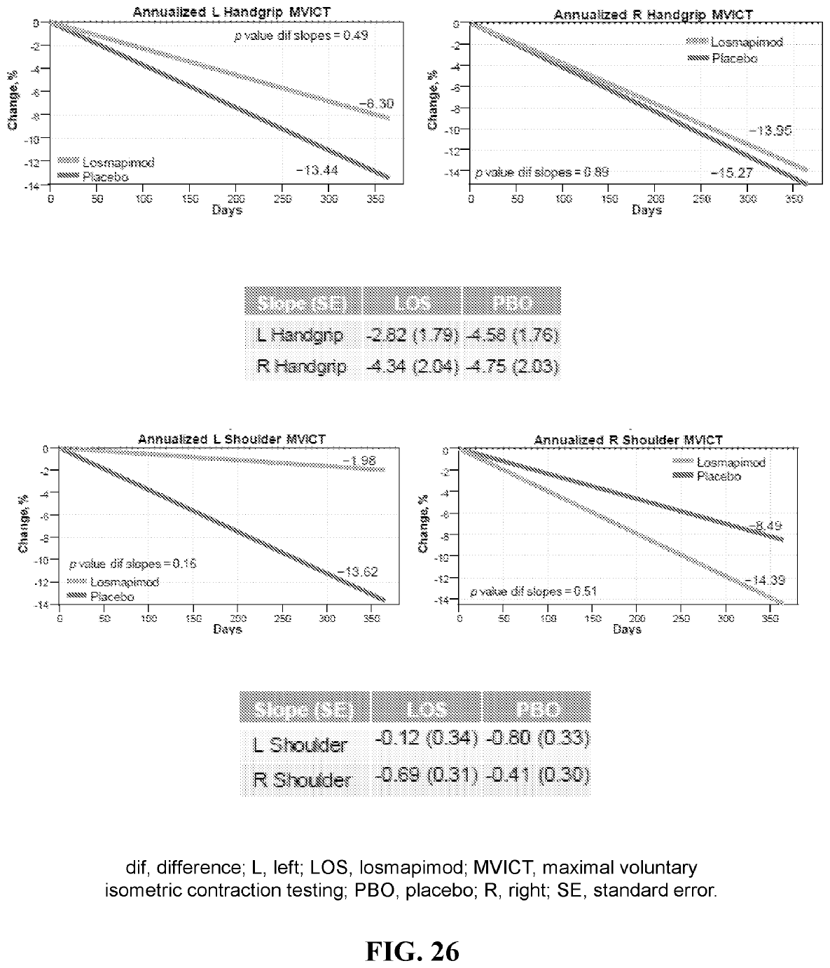

FIG. 23 shows that losmapimod shows slowing of disease progression or improvement in function across all domains, most noticeably in the above the shoulder domains (Q1 and Q3). Further, annualized rates of change in MFI, annualized rates of change in MFF, and annualized rates of change in LMV are shown in FIG. 24. Annualized rates of change in TUG Average Completion Time are shown in FIG. 25. Annualized rates of change in maximal voluntary isometric contraction testing (MVICT) for handgrips and shoulders are shown in FIG. 26.

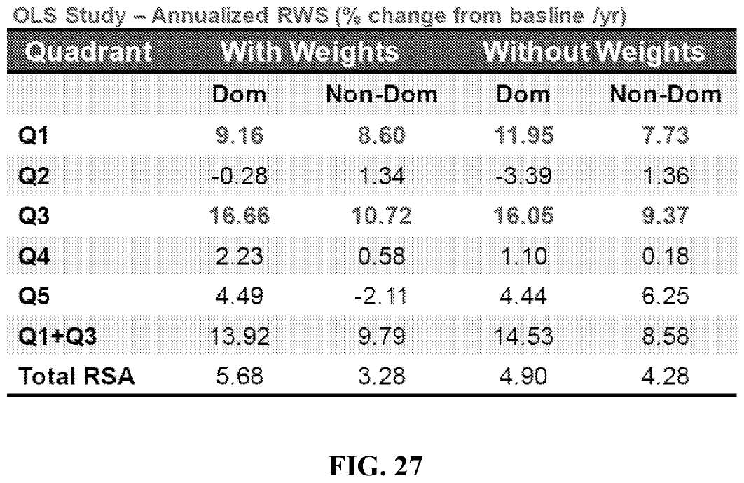

In the Open Label Study, reachable workspace (RWS) with and without weights has also been evaluated. FIG. 27 provides exploratory annualized percentage changes of RWS from baseline of patients participating in the study. The data show that annualized RWS increased for all quadrants in the Open Label Study, with the largest increases in Q1 and Q3 (7-17%).

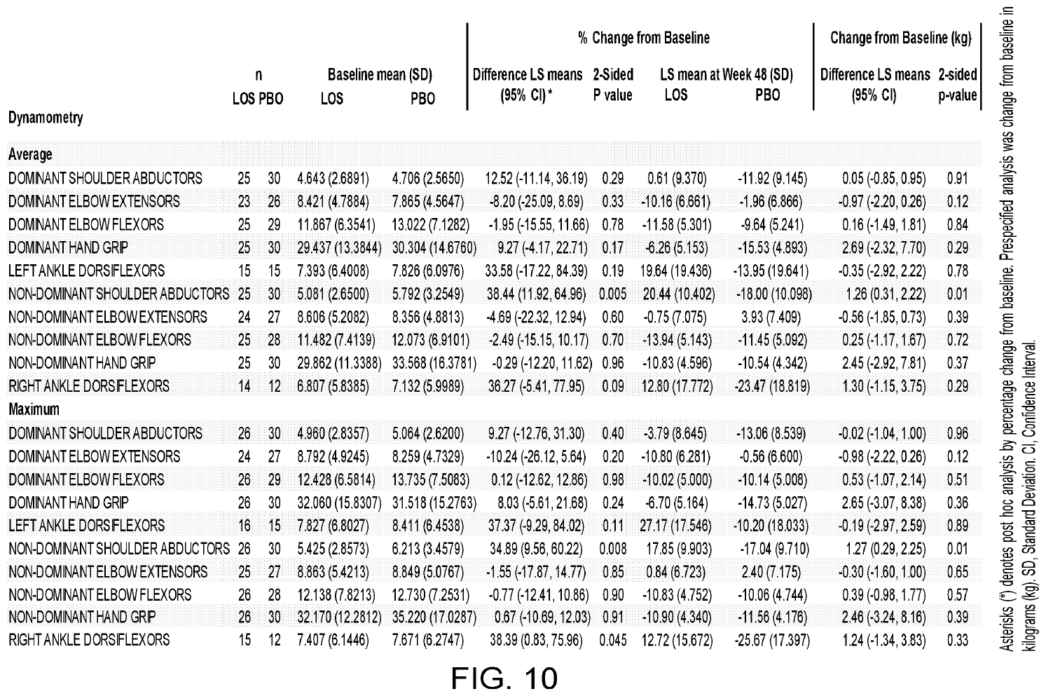

Assessment of maximum voluntary isometric contraction (MVICT) was conducted with a hand-held dynamometer. Post-hoc analyses showed the strength of shoulder abductors and ankle dorsiflexors in the losmapimod group with less worsening (dominant shoulder −3.79%, ns), or improvements (non-dominant shoulder 17.85%, difference 34.89; p<0.01; right ankle 12.72%, difference 38.39; p<0.05; left ankle 27.17%, difference 37.37; p=0.11) (FIG. 6). Subjects in the placebo group lost strength in the shoulder abductors (dominant, −13.1%, non-dominant −17.0%) and ankle dorsiflexors (right ankle, −25.7%, left ankle 10.20%) at Wk48 (FIG. 10). Assessment of the average of three repeated trials was consistent with the MVICT showing improvement or preservation of bilateral shoulder abductors and ankle dorsiflexors. No difference was observed in either average or MVICT in other muscle groups including bilateral hand grip, elbow flexors or extensors (FIG. 10).

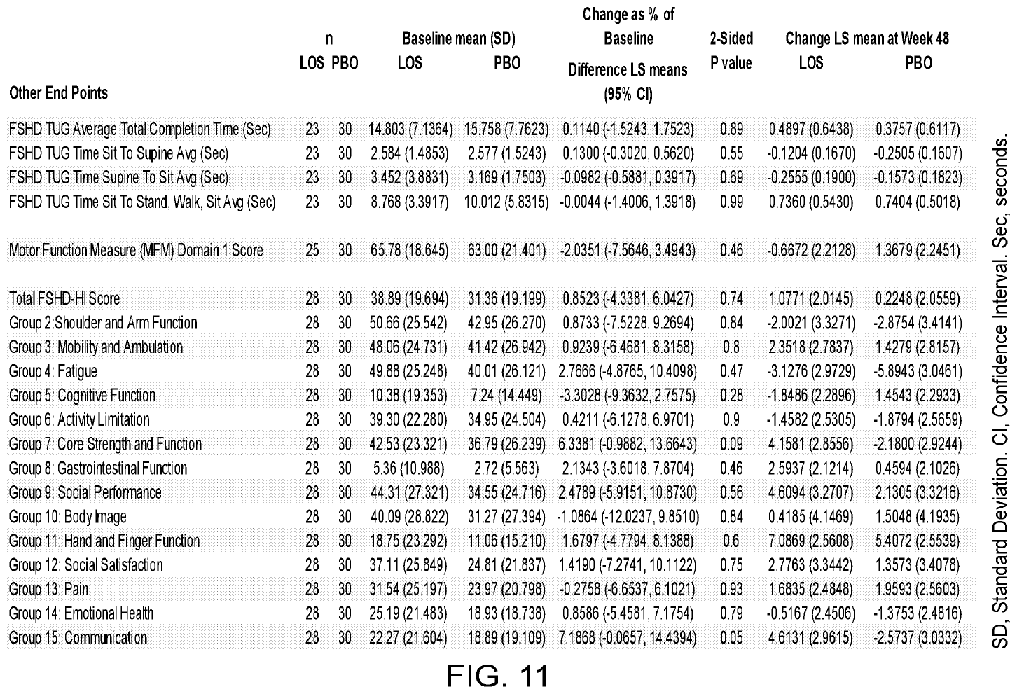

Losmapimod subjects had a nonsignificant, but clinically meaningful, slowing of progression in TUG time (0.16 s vs. 0.74 s; difference 0.58 s) (FIG. 6). The FSHD-TUG, MFM total score, and FSHD-HI showed no difference. (FIG. 6 and FIG. 11).

In this trial, losmapimod demonstrated clinical benefit in multiple clinical outcome assessments (COAs) and WB-MSK-MRI measures consistent with the hypothesis that losmapimod may slow FSHD progression. Losmapimod continued to demonstrate a favorable safety and tolerability consistent with that observed in >3500 subjects previously dosed.

WB-MSK-MRI captured the heterogeneity of the disease and provided important information about disease severity as it correlates with FSHD-relevant clinical endpoints. The MRI composites demonstrated sensitivity to disease progression by correlating with FSHD relevant COAs in cross-sectional analyses. MFI has been shown to be a highly sensitive, proximal biomarker for loss of muscle function in sarcopenia, and this study implies the same for FSHD. After 48 weeks, reduction in the progression of MFI and MFF in A and B muscles suggests losmapimod has an impact on fat accumulation in muscles that have not yet reached end-stage which has been previously reported to have little remaining functional capacity.

As suggested by the preservation of muscle structure measured by MRI, treatment with losmapimod resulted in slowing of progression or improvement in FSHD-relevant clinical assessments. Improvement of shoulder function assessed by RWS, a COA with high reliability, repeatability, face validity, feasibility, and sensitivity to change that is highly correlated to activities of daily living. This finding is corroborated by preservation or improvement in shoulder abduction strength on dynamometry. Additionally, improvements in ankle dorsiflexion strength are supportive of the trends of faster TUG times. The slowing of progression or improvement in these COAs projected over time via annualized rates of change is evidence losmapimod favorably alters disease progression in FSHD.

These improvements are recognized by subjects (clinically meaningful difference of 0.58 on PGIC between losmapimod and placebo groups). Nearly 20% of treated subjects reported perceived improvement on the PGIC, while many placebo subjects reported perceived decline.

Even though there is downstream evidence of benefits of DUX4 reduction, the prespecified population and subgroup analyses did not show differences in DUX4-driven gene expression, between or within groups at Wk16 or Wk36. Based on the target engagement observed in preclinical studies, 30-70% decreases in DUX4-driven gene expression of similar magnitude was hypothesized. At a population level, DUX4-driven gene expression spanned over 1,000-fold between subjects at baseline, demonstrating large variance. Multiple factors contributed to this large variability, including the stochastic nature of DUX4-driven gene expression, the very transient duration of and relative scarcity of DUX4 expression among myonuclei (which has been hypothesized to be ˜ 1/1000- 1/3000), the heterogeneity in composition of FSHD muscles, and the relative imprecision in the biopsy procedure.

### Example 2. A Phase 3 Randomized, Double-Blind, Placebo-Controlled, 48-Week Study of the Efficacy and Safety of Losmapimod in FSHD

The objective of this study will be to evaluate the efficacy and safety of losmapimod for the treatment of FSHD. A total of approximately 230 patients, 210 patients with genetically confirmed FSHD1 and 20 patients with FSHD2, will be randomized 1:1 to receive losmapimod or placebo orally, twice daily for 48-weeks. Efficacy assessments include reachable workspace quantification of total relative surface area (Q1-Q5) with 500 g wrist weight in the dominant arm, evaluation of quality of life in the neurological disorders upper extremity scale (Neuro-QOL UE), patient global impression of change (PGIC), and muscle fat infiltration (MFI) on whole-body musculoskeletal MRI (WB-MSK MRI). Exploratory assessments include muscle fat fraction, assessment of muscle strength by hand-held dynamometry, and patient reported outcomes (PRO) including patient global impression of severity (PGIS), novel FSHD PRO, numeric pain rating scale (NPRS), 5-level EQ-5D (EQ-5D-5L) and healthcare utilization questionnaire.

### Example 3. Muscle Ultrasound in an Open-Label Study of Losmapimod in Subjects with FSHD1

Fourteen subjects age 18 to 65 years with genetically confirmed FSHD1, clinical severity score of 2 to 4 (range 0-5) and MRI-eligible skeletal muscles for needle biopsy received open-label 15 mg twice daily losmapimod for 52 weeks with the primary objective of safety. Assessments included safety, MRI, muscle ultrasound (US), clinical outcomes, and PROs. US was performed on 7 muscles bilaterally using a standardized protocol. US echointensity was expressed as a z-score relative to matched healthy controls, with abnormal being defined as >2.

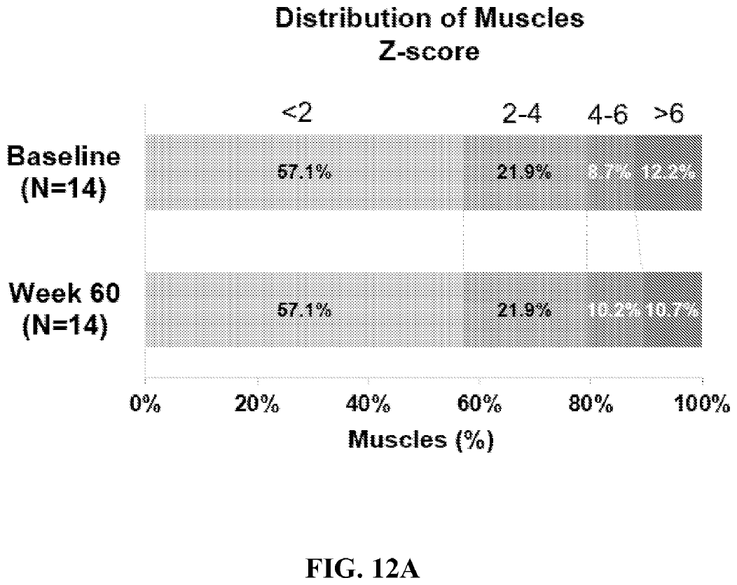

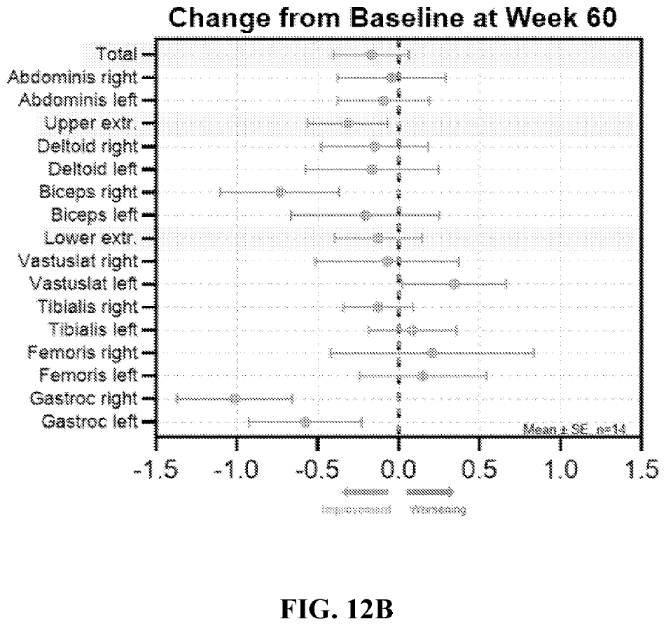

The mean (SD) change from baseline in echogenicity of all muscles was-0.17 (0.9), in the upper extremity muscles −0.32 (0.9) and lower extremities −0.13 (1.0), representing a trend towards improvement. The distribution of the z-scores (<2, 2-4, 4-6, and >6) of muscles at baseline versus the last visit stayed the same or decreased, consistent with stability. Distributions are shown in more detail in FIG. 12A. Further, most muscles demonstrated stability or improvement over 52 weeks of treatment (FIG. 12B). Natural history studies previously demonstrated increases or worsening in echogenicity over 1 year. Echointensity correlated strongly with muscle fat infiltration (MFI) for the biceps brachii (r=0.84, p<0.01), tibialis anterior (r=0.76, p<0.01), and gastrocnemius medialis (r=0.50, p<0.01). A ceiling effect of echogenicity observed for at MFI greater than about 10% for Biceps Brachii and greater than about 20% for Tibialis Anterior and Quadriceps. Further, Echointensity appears to falsely normalize in more severely fat replaced muscle, Gastrocnemius Medialis, at an MFI greater than 25%, due to the tissue becoming homogenous (fat).

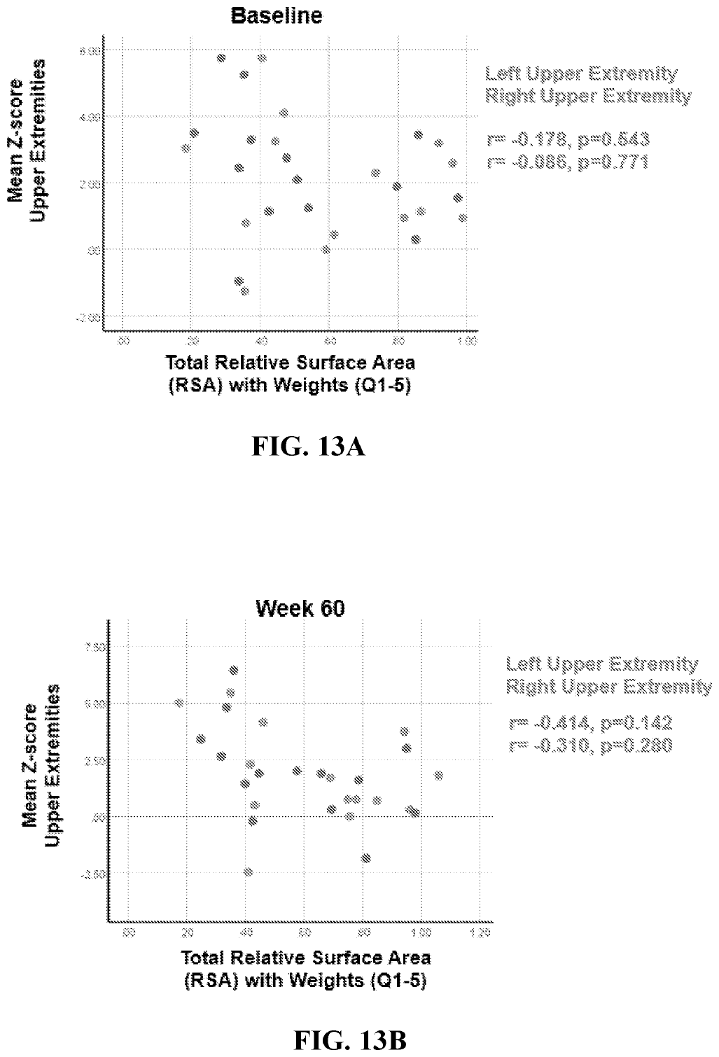

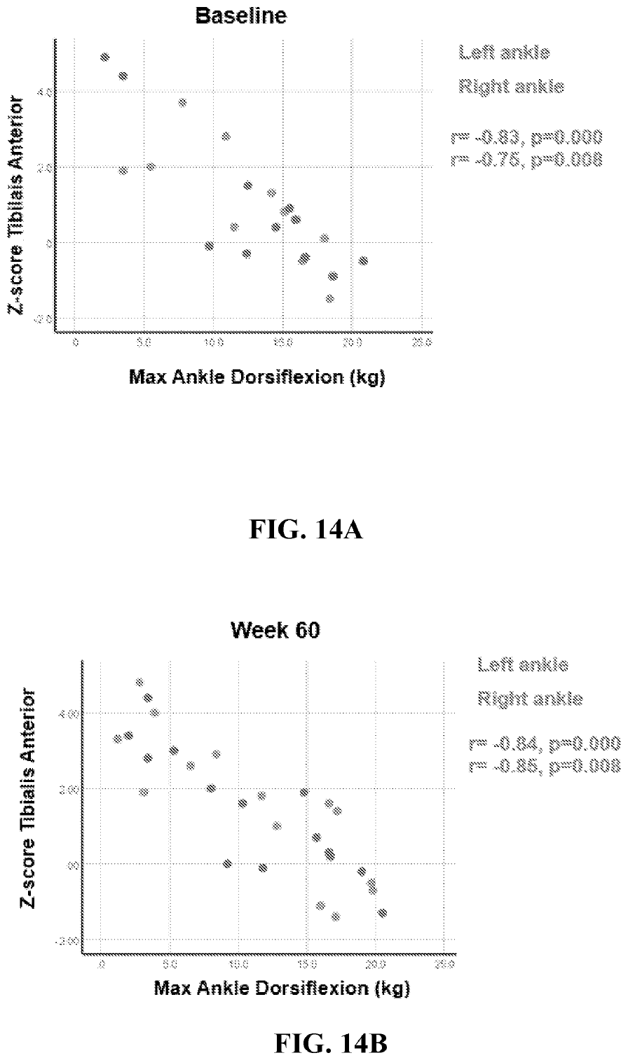

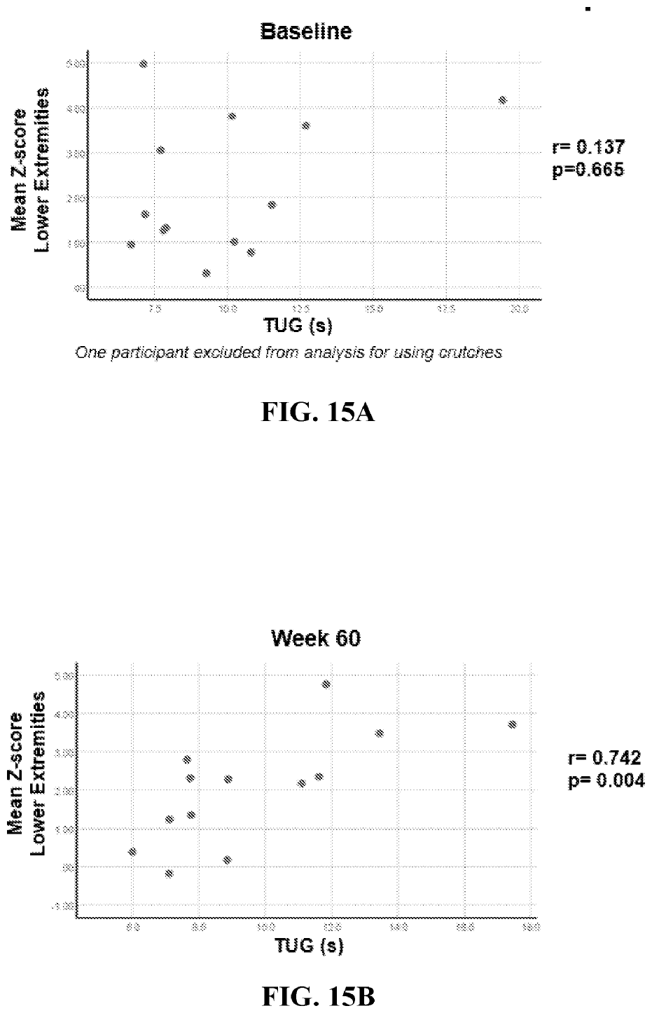

Correlation with Clinical Outcome Assessments (COAs) were also evaluated. In one example Reachable Workspace data at Baseline and Week 60 are provided in FIG. 13A (Baseline) and FIG. 13B (Week 60), which shows a non-significant trend of correlation between Total Weighted RSA and upper extremity echogenicity (biceps and deltoids). In another example Handheld Dynamometry for Ankle Dorsiflexion data at Baseline and Week 60 are provided in FIG. 14A (Baseline) and FIG. 14B (Week 60), which shows a strong correlation of echogenicity of Tibialis Anterior to maximum ankle dorsiflexion. In another example Timed Up-And-Go (TUG) data at Baseline and Week 60 are provided in FIG. 15A (Baseline) and FIG. 15B (Week 60), which shows a mean echogenicity of the lower extremities showed strong correlation to the classic TUG at Week 60. Correlation at baseline limited by outlier on TUG.

### Example 4. Feasibility of Measuring Functional Performance of FSHD Patients Using Wearable Sensors to Quantify Physical Activity

This study evaluated feasibility to monitor daily activity and assess functional outcomes using wearable sensor devices in an open label study (OLS) of losmapimod in facioscapulohumeral muscular dystrophy (FSHD). 14 patients with FSHD1 received 15 mg losmapimod twice per day for a 52 week open label treatment period.

Feasibility was measured by the reliability of the data over time and amount of background noise. The secondary objective was patients' compliance with using the wearable device.

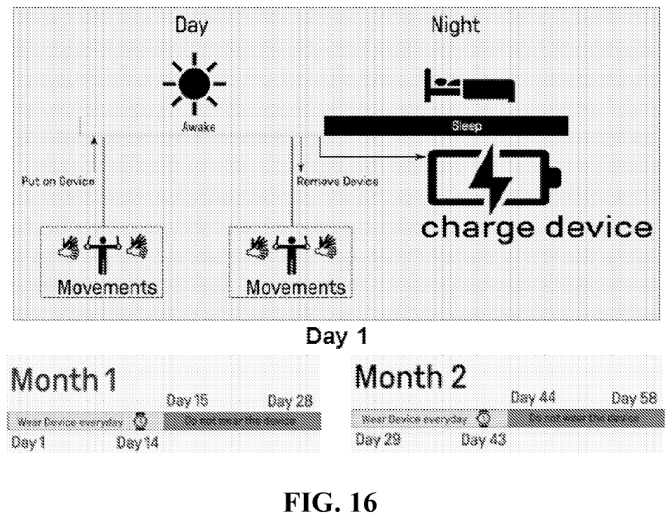

Upper limb and lower limb mobility were evaluated with “Actimyo” made by Sysnav. Actimyo quantifies movements of patients with neuromuscular disorders such as Duchenne muscular dystrophy (DMD), Spinal amyotrophy (SMA), Parkinson's and records activity based on magneto-inertial navigation. Raw data from the sensors are loaded on to the internal memory of the docking station/charging unit, and transmitted daily via an internet connection to a remote storage platform. Participants are provided with two watch like sensors to measure movements: On the ankle for lower extremity movements like gait; and On the wrist for upper extremity movements like mobility. Two kinds of movements were evaluated: Free living movement, which was recorded by wearing two devices all day (12-16 hours); and Imposed movements (2 times per day), which were a set of movements evaluated twice daily. A schematic of a patient's use of the device is shown in FIG. 16.

### Results:

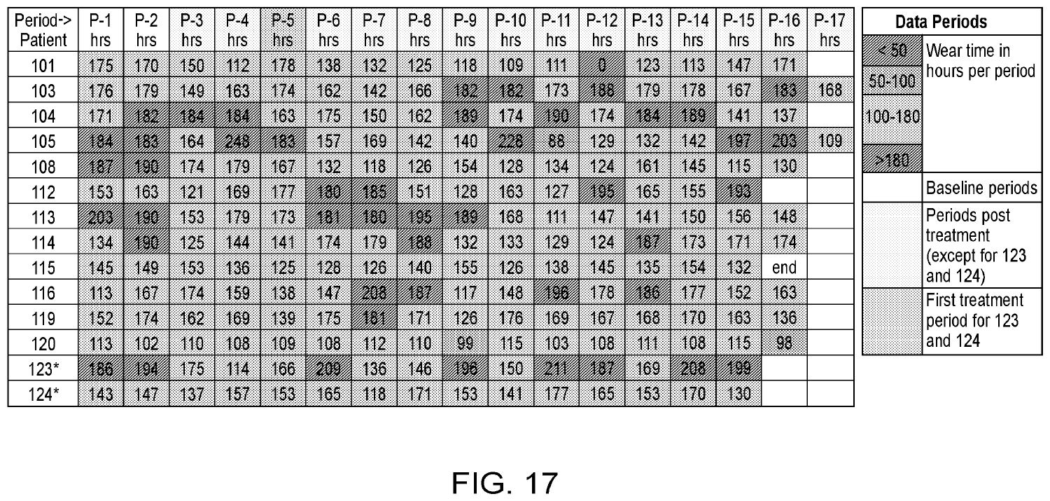

Feasibility and Compliance: Participant compliance for wearing the devices was at 99%. The total number of days that all 14 participants were monitored was 2,941 days or 36,758 hours (an average of 2,626 hours per participant). Results for patients by period are in FIG. 17.

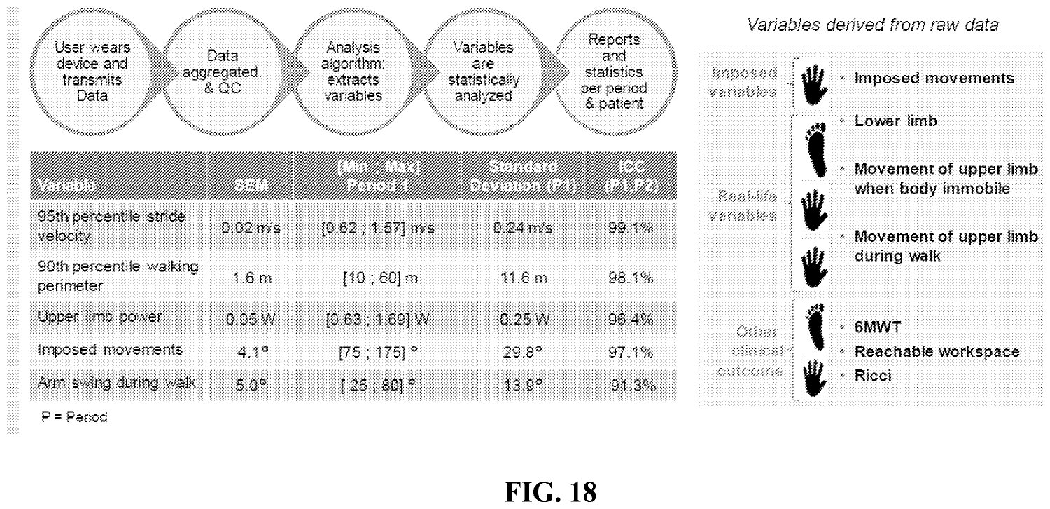

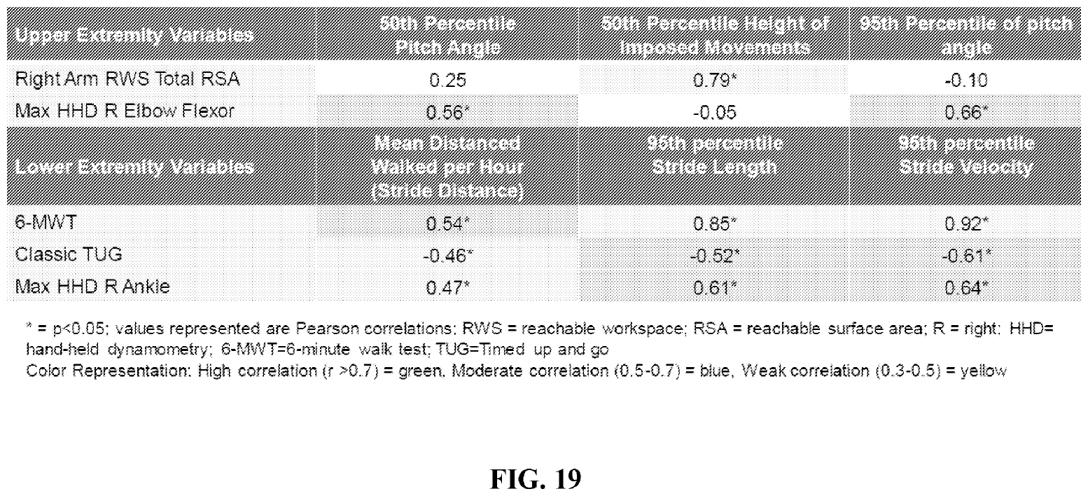

Analysis, processing and reliability results are presented in FIG. 18. Moderate to strong correlations between clinic and wearable variables at Baseline are also shown in FIG. 19.

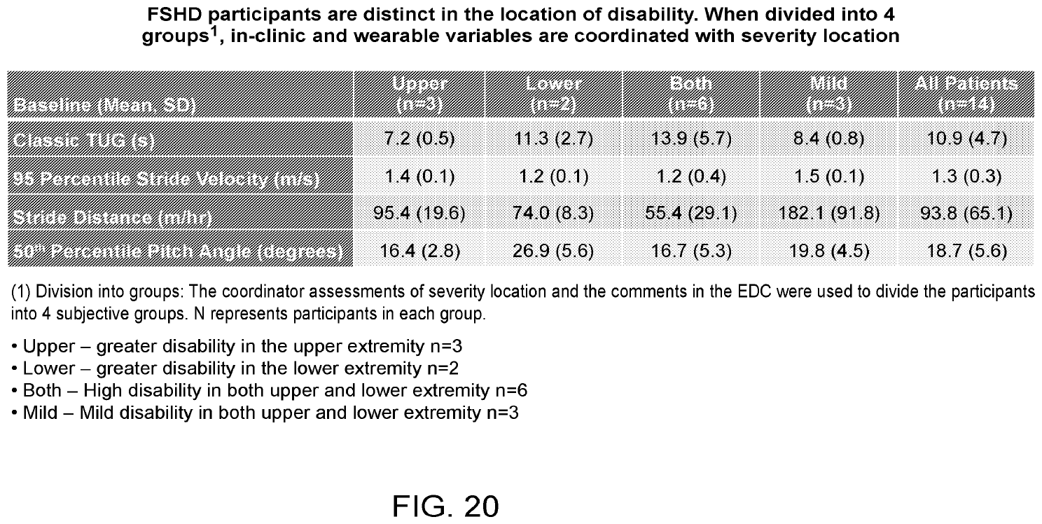

FSHD participants are distinct in the location of disability. When divided into 4 groups, in-clinic and wearable variables are coordinated with severity location, results of which are depicted in FIG. 20.

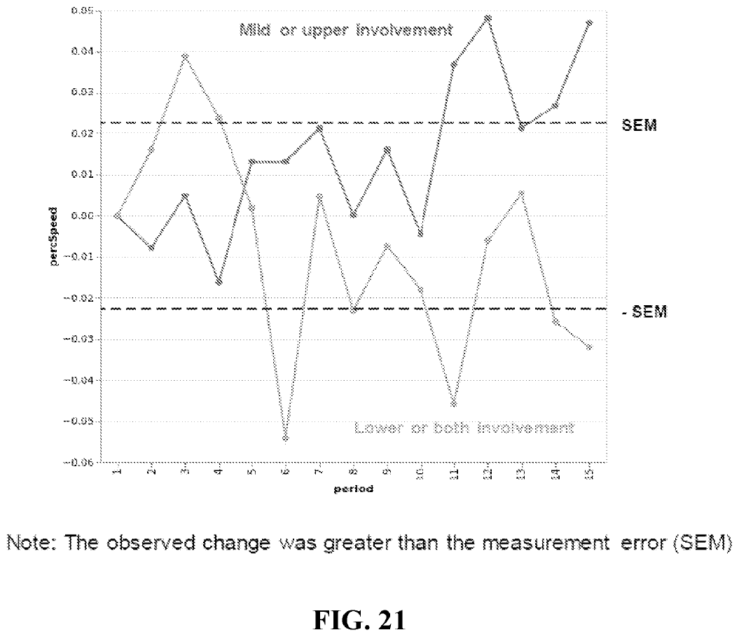

Furthermore, physical function as measured by gait velocity tends to increase over 1 year treatment period in “Mild” or “Upper” group as compared to “Lower” or “Both” group, which is further shown in FIG. 21.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of treating facioscapulohumeral muscular dystrophy (FSHD) in a patient in need thereof, comprising:
administering 15 mg, twice daily, of losmapimod to the patient for at least 40 weeks,
wherein after the at least 40 weeks the patient has improved muscle health as measured by whole-body musculoskeletal magnetic resonance imaging (WB-MSK-MRI).

2. A method of reducing fat accumulation in a muscle at a high risk of progression of facioscapulohumeral muscular dystrophy (FSHD), in a patient in need thereof, comprising:
administering 15 mg, twice daily, of losmapimod for at least 40 weeks.

3. The method of claim 1 or 2, wherein after the at least 40 weeks the muscle has reduced fat accumulation as measured by whole-body musculoskeletal magnetic resonance imaging (WB-MSK-MRI).

4. The method of claim 2 or 3, comprising imaging the muscle using whole-body musculoskeletal magnetic resonance imaging (WB-MSK-MRI) to determine a correlation between a measure of muscle health and a clinical outcome assessment of the patient.

5. The method of claim 4, wherein the measure of muscle health is selected from the group consisting of muscle fat fraction (MFF), muscle fat infiltration (MFI), and lean muscle volume (LMV).

6. The method of claim 4, wherein the clinical outcome assessment is selected from the group consisting of a reachable workspace (RWS), a relative surface area (RSA), and an assessment made from a Timed Up and Go (TUG) test.

7. The method of any one of claims 2-7, wherein the muscle, prior to administering losmapimod to the patient, is characterized by a muscle fat infiltration (MFI) of less than about 0.10 and a muscle fat fraction (MFF) of less than about 0.50.

8. The method of any one of claims 2-7, wherein the muscle, prior to administering losmapimod to the patient, is characterized by a muscle fat infiltration (MFI) of greater than or equal to about 0.10 and a muscle fat fraction (MFF) of less than about 0.50.

9. The method of any one of claims 1-8, comprising administering 15 mg, twice daily, of losmapimod to the patient for at least 48 weeks.

10. The method of any one of claims 1-9, wherein the FSHD is FSHD1.

11. The method of any one of claims 1-10, wherein the muscle is selected from the group consisting of a muscle of an upper extremity and a muscle of a lower extremity.

12. The method of any one of claims 1-10, wherein the muscle is selected from the group consisting of shoulder abductor and an ankle dorsiflexor.

13. The method of claim 12, wherein the shoulder abductor is selected from the group consisting of a bilateral shoulder abductor, a dominant shoulder abductor, and a non-dominant shoulder abductor.
